(19) United States
(12) Patent Application Publication
(10) Pub. No.: US 2009/0196919 A1
(43) Pub. Date: Aug. 6, 2009

(54) OXCARBAZEPINE DOSAGE FORMS

(76) Inventors: Ajay Singla, Chandigarh (IN); Sachin Arora, Uttar Pradesh (IN); Rahul Dabre, Haryana (IN)

Correspondence Address: RANBAXY INC.;INTELLECTUAL PROPERTY DEPT., 600 COLLEGE ROAD EAST, SUITE 2100, PRINCETON, NJ, 08540, US

(21) Appl. No.: 11/577,902

(22) PCT Filed: Oct. 24, 2005
(86) PCT No.: PCT/IB05/03168

§ 371 Date: Apr. 1, 2009

(30) Foreign Application Priority Data
Oct. 25, 2004 (IN) 2084/DEL/2004

(51) Int. Cl. A61K 9/48; C07D 223/22; A61K 31/55; A61K 9/14; A61K 9/20; A61P 25/08

(52) U.S. Cl. 424/452; 540/589; 514/217; 424/489; 424/465

(57) ABSTRACT

The present invention relates to dosage forms of oxcarbazepine for oral administration that contain oxcarbazepine having a median particle size of from about 14 μm to about 30 μm and to processes for the preparation of such dosage forms. The dosage form may be a solid or a liquid dosage form. The solid dosage form may be in the form of a tablet, a capsule, or a granulate. The liquid dosage form may be in the form of a solution or a suspension.

### FIELD OF THE INVENTION

The present invention relates to dosage forms of oxcarbazepine for oral administration and to processes for the preparation of such dosage forms.

### BACKGROUND OF THE INVENTION

Drug insolubility is one of the major challenges in the development of many pharmaceutical products. Over one third drugs listed in the US Pharmacopoeia and about fifty percent of New Chemical Entities are insoluble or poorly soluble in water. The result is that many drugs are marketed as sub-optimal formulations, which after administration lead to poor or erratic bioavailability or a greater risk of adverse side effects. Oxcarbazepine, 10,11-dihydro-10-oxo-5H-dibenz[b,f]azepine-5-carboxamide, a widely used antiepileptic drug has poor solubility in water.

One of the earlier attempts to enhance the dissolution rate and bioavailability of oxcarbazepine relied on particle size reduction of the oxcarbazepine to an order of 2 to 12 μm. International (PCT) Publication No. WO 98/35681 discloses a composition of oxcarbazepine for oral administration employing micronized drug particles of 2 to 12 μm ranges.

Oxcarbazepine tablets are also known to undergo a color change during storage. The discoloration is caused by the formation of a minor amount of an oxidation product “diketoiminodibenzyl”, 10,11-dihydro-5H-dibenzo[b,f]azepine-10,11-dione. This oxidation product is considered to be pharmacologically harmless. However, the color change is not generally pharmaceutically desirable.

U.S. Pat. Nos. 5,472,714 and 5,695,782 describe color stable oxcarbazepine tablets. The color stability has been achieved by providing double coating to the tablets. Oxcarbazepine tablets described therein are provided with hydrophilic, permeable inner layer containing white pigments and further a hydrophilic, permeable outer layer containing white pigments in combination with iron (II) oxide pigments.

Our International (PCT) Publication No. WO 02/094774, which is hereby incorporated by reference, discloses an oxcarbazepine formulation comprising oxcarbazepine and a wetting agent.

### SUMMARY OF THE INVENTION

In one general aspect there is provided dosage forms that include oxcarbazepine having a median particle size of from about 14 μm to about 30 μm. In particular, the median particle size may be from about 14 μm to about 25 μm.

The dosage form may be a solid or a liquid dosage form. The solid dosage form may be in the form of a tablet, a capsule, or a granulate. The liquid dosage form may be in the form of a solution or a suspension.

Embodiments of the dosage form may include one or more of the following features. For example, the solid dosage form may further include one or more pharmaceutically acceptable excipients that include surfactants, diluents, binders, disintegrants, lubricants, glidants, and coloring agents. The liquid dosage form may further include one or more pharmaceutically acceptable excipients that include surfactants, diluents, binders, disintegrants, lubricants, glidants, and coloring agents.

In another general aspect there is provided a process for preparing a solid dosage form of oxcarbazepine. The process includes mixing the oxcarbazepine having a median particle size of from about 14 μm to about 30 μm with other pharmaceutical excipients to form a blend and forming the blend into a solid dosage form.

Embodiments of the process may include one or more of the following features. For example, in the process, shaping of the blend into a solid dosage form may include forming a tablet, capsule, or granulate.

The mixing may be one or more of wet granulation, dry granulation, and direct compression. The solid dosage form may include one or more pharmaceutically acceptable excipients selected from surfactants, diluents, binders, disintegrants, lubricants, glidants, and coloring agents.

In another general aspect there is provided a process for preparing a liquid dosage form of oxcarbazepine. The process includes dispersing a suspending agent and oxcarbazepine having a median particle size of from about 14 μm to about 30 μm; and homogenizing. The suspending agent may be one or more of polysaccharides, a mixture of cellulose and xanthan gum, a mixture of polyethylene glycol and sodium carboxymethyl cellulose, a mixture of xanthan gum and pregelatinized starch, a mixture of microcrystalline cellulose and sodium carboxymethyl cellulose (Avicel RC 591), and dispersed silicon dioxide (Aerosil 200).

Embodiments of the process may include one or more of the following features. For example, the dosage form may further include one or more pharmaceutically acceptable excipients that include surfactants, diluents, binders, disintegrants, lubricants, glidants, and coloring agents.

In another general aspect there is provided a method of treating partial seizures in adults with epilepsy and as an adjunct therapy for treating partial seizures in children ages 4-16 with epilepsy. The method includes orally administering to a human in need thereof a dosage form that includes the oxcarbazepine having a median particle of from about 14 μm to about 30 μm.

Embodiments of the dosage form may include one or more of the following features. For example, the solid dosage form may further include one or more pharmaceutically acceptable excipients that include surfactants, diluents, binders, disintegrants, lubricants, glidants, and coloring agents. The liquid dosage form may further include one or more pharmaceutically acceptable excipients that include surfactants, diluents, binders, disintegrants, lubricants, glidants, and coloring agents.

The details of one or more embodiments of the inventions are set forth in the description below. Other features, objects and advantages of the inventions will be apparent from the description and claims.

### DETAILED DESCRIPTION OF THE INVENTION

We have now discovered that stable and bioequivalent oxcarbazepine dosage forms can be prepared with oxcarbazepine having a median particle size of from about 14 μm to about 30 μm.

Oxcarbazepine, 10,11-dihydro-10-oxo-5H-dibenz[b,f]azepine-5-carboxamide is an agent of first choice in the treatment of convulsions. The known dosage forms, such as tablets and liquid dosage forms, for example suspensions, are suitable for ensuring a uniform concentration of active ingredient in the blood, especially in the case of regularly recurring administration over a prolonged period of treatment.

The known particle size analysis methods are suitable for determining the median particle size, for example particle size measurement using light, for example light-scattering methods or turbidimetric methods, sedimentation methods, for example pipette analysis using an Andreassen pipette, sedimentation scales, photosedimentometers or sedimentation in a centrifugal force field, pulse methods, for example using a Coulter counter, or sorting by means of gravitational or centrifugal force.

In order to produce oxcarbazepine particles, for example crystals having the desired particle size, conventional comminution and de-agglomeration techniques may be used, for example grinding in an air-jet mill or impact mill, a ball mill, vibration mill, mortar mill or pin mill.

The pharmaceutically acceptable excipients may be selected from one or more of surfactants, diluents, binders, disintegrants, lubricants, glidants, suspending agents, solvents, antioxidants, preservatives, coloring agents, flavoring agents and sweeteners, which are chemically and physically compatible with oxcarbazepine.

The surfactant may be selected from anionic, cationic or non-ionic surface-active agents or surfactants. Suitable anionic surfactants include those containing carboxylate, sulfonate, and sulfate ions such as sodium lauryl sulfate (SLS), sodium laurate, dialkyl sodium sulfosuccinates particularly bis-(2-ethylhexyl) sodium sulfosuccinate, sodium stearate, potassium stearate, sodium oleate and the like. Suitable cationic surfactants include those containing long chain cations, such as benzalkonium chloride, bis-2-hydroxyethyl oleyl amine or the like. Suitable non-ionic surfactants include polyoxyethylene sorbitan fatty acid esters, fatty alcohols such as lauryl, cetyl and stearyl alcohols; glyceryl esters such as the naturally occurring mono-, di-, and tri-glycerides; fatty acid esters of fatty alcohols; polyglycolized glycerides such as Gelucire; polyoxyethylene-polyoxypropylene block co-polymer such as Poloxamer and other alcohols such as propylene glycol, polyethylene glycol, sorbitan, sucrose, and cholesterol.

Diluents may be selected from any such pharmaceutically acceptable excipients that gives bulk to the oxcarbazepine composition and improves compressibility. For example, preferable diluents include one or more of calcium carbonate, calcium phosphate-dibasic, calcium phosphate-tribasic, calcium sulfate, cellulose-microcrystalline, cellulose powdered, dextrates, dextrins, dextrose excipients, fructose, kaolin, lactitol, lactose, mannitol, sorbitol, starch, starch pregelatinized, sucrose, sugar compressible, or sugar confectioners.

Binders may be selected from any pharmaceutically acceptable excipients that have cohesive properties to act as a binder. For example, preferably excipients include one or more of methyl cellulose, hydroxypropyl cellulose, hydroxypropyl methylcellulose, polyvinylpyrrolidone, gelatin, gum arabic, ethyl cellulose, polyvinyl alcohol, pregelatinized starch, agar, tragacanth, sodium alginate, or propylene glycol.

The disintegrant may be selected from, for example, one or more of starches or modified starches such as starch, modified starch, croscarmellose sodium, crospovidone and sodium starch glycolate. Other suitable disintegrants also may be used separately or in combination.

Lubricants may be selected from, for example, one or more of colloidal silicon dioxide, stearic acid, magnesium stearate, calcium stearate, talc, hydrogenated vegetable oil, sucrose esters of fatty acid, microcrystalline wax, yellow beeswax, white beeswax, glyceryl monostearate, and PEG 4000. Other suitable lubricants also may be used separately or in combination.

Glidants may be selected from, for example, colloidal silicon dioxide and talc, although any other suitable glidants may be used.

Suspending agents may be selected from, for example, one or more of polysaccharides, a mixture of cellulose and xanthan gum, a mixture of polyethylene glycol and sodium carboxymethyl cellulose, a mixture of xanthan gum and pregelatinized starch, a mixture of microcrystalline cellulose and sodium carboxymethyl cellulose (Avicel RC 591), or dispersed silicon dioxide (Aerosil 200). The polysaccharides can be selected from one or more of tragacanth, xanthan gum, bentonite, acacia and lower alkyl ethers of cellulose including the hydroxy and carboxy derivatives of the cellulose ethers.

The dosage form may further include antioxidants to protect the oxcarbazepine from oxidative degradation. The antioxidants may be selected from, for example, ascorbic acid, sodium pyrosulphite, glutathion or sorbic acid.

The suspension for oral administration is usually aqueous based. The term “aqueous based” as used herein includes suspensions comprising water, or water and one or more of water-miscible solvents. Suitable water miscible solvents include, but not limited to, propylene glycol, polyethylene glycol, ethanol and other commonly used solvents known to the skilled in the art. These solvents also act as solvents for preservatives.

Examples of preservatives include propylparaben, methylparaben, and sorbic acid, sodium benzoate, or sodium bisulphate.

Coloring agents may be selected from any colorant used in pharmaceuticals that is approved and certified by the FDA. It may include Iron oxide, Lake of Tartrazine, Lake of Quinoline Yellow, Lake of Sunset Yellow and Lake of Erythrosine, Lack of Carmosine Ponceau, Allura Red.

Sweeteners may be selected from sucrose, lactose, glucose, aspartame, saccharine, or sorbitol solution.

Examples of suitable flavoring agents include yellow plum lemon, aroma, peppermint oil, oil of wintergreen, cherry, orange or raspberry flavoring.

The dosage forms that include oxcarbazepine, and other excipients include tablets, caplets, capsules, granules, suspension and solution. The dosage forms of oxcarbazepine can be conveniently prepared by any of the methods known to those skilled in the art. For tablets, the method of choice may be wet granulation, dry granulation or direct compression. These methods include the basic step of intimately mixing the oxcarbazepine having a median particle size of from about 14 μm to about 30 μm with other pharmaceutically acceptable excipients and shaping the product into a solid dosage form. Alternatively, the wet granulation may be carried out by granulating fluid or a solution or dispersion of surfactant or solution or suspension of binder.

The granulating liquid can be, but is not limited to, water, ethanol, isopropyl alcohol, acetone, dichloromethane, and the like. Alternatively, the binder can be dissolved in the granulating liquid and used as a solution/dispersion.

The tablet dosage form may optionally be coated with functional and/or non-functional layers comprising film-forming polymers. The coating composition may include polymers and other coating additives.

Examples of film-forming polymers include ethylcellulose, hydroxypropyl methylcellulose, hydroxypropylcellulose, methylcellulose, carboxymethyl cellulose, hydroxymethylcellulose, hydroxyethylcellulose, cellulose acetate, hydroxypropyl methylcellulose phthalate, cellulose acetate phthalate, cellulose acetate trimellitate; waxes such as polyethylene glycol; methacrylic acid polymers such as Eudragit® RL and RS; and the like. Alternatively, commercially available coating compositions comprising film-forming polymers marketed under various trade names, such as Opadry® may also be used for coating.

Coating additives may be selected from, for example, plasticizers, coloring agents, gloss producer, lubricants/glidants.

Polymer solution or dispersion may be prepared in various solvents such as water, ethanol, methanol, isopropyl alcohol, chloroform, acetone, ether or mixtures thereof. The coating composition can be coated onto solid dosage form using techniques such as spray coating in conventional coating pan or fluidized bed processor, or dip coating.

The invention described herein is further illustrated by the following examples, which should not be construed as limiting the scope of the invention.

### Example 1

| | Quantity |
|---|---|
| Ingredient | (mg/unit) |
| Oxcarbazepine | 600.0 |
| (Median particle size 20.86 μm) | |
| Microcrystalline cellulose | 131.2 |
| Hydroxypropyl methyl cellulose | 16.8 |
| Cross-linked polyvinylpyrrolidone | 40.0 |
| Colloidal silicon dioxide | 3.2 |
| Magnesium stearate | 8.8 |
| Total | 800.0 |

### Process:

1. Microcrystalline cellulose (about half the quantity) and hydroxy propyl methylcellulose were sifted through sieve; and mixed with oxcarbazepine for about 10 minutes to make a uniform blend.
2. Dry blend of step 1 was granulated with water.
3. The wet mass of step 2 was dried in fluidized bed dryer.
4. The dried material of step 3 was passed through sieve.
5. Cross-linked polyvinylpyrrolidone, colloidal silicon dioxide and microcrystalline cellulose (rest of the quantity) were sieved. These were then mixed with the dried material of step 4.
6. Magnesium stearate was passed through sieve and mixed with the material of step 5.
7. Lubricated blend of step 6 was compressed.

### Examples 2

| | Quantity |
|---|---|
| Ingredient | (mg/unit) |
| Oxcarbazepine | 600.0 |
| (Median particle size 15 μm) | |
| Microcrystalline cellulose | 131.2 |
| Hydroxypropyl methyl cellulose | 16.8 |
| Sodium lauryl sulphate | 20.0 |
| Cross-linked | 40.0 |
| polyvinylpyrrolidone | |
| Colloidal silicon dioxide | 3.2 |
| Magnesium stearate | 8.8 |
| Total | 800.0 |

### Process:

1. Microcrystalline cellulose (about half the quantity) and hydroxy propyl methylcellulose were sifted through sieve; and mixed with oxcarbazepine for about 10 minutes to make a uniform blend.
2. Sodium lauryl sulphate was dissolved in water and dry blend of step 1 was granulated.
3. The wet mass of step 2 was dried in fluidized bed dryer.
4. The dried material of step 3 was passed through sieve.
5. Cross-linked polyvinyl pyrrolidone, colloidal silicon dioxide and microcrystalline cellulose (rest of the quantity) were sieved. These were then mixed with the dried material of step 4.
6. Magnesium stearate was passed through sieve and mixed with the material of step 5.
7. Lubricated blend of step 6 was compressed.

### Example 3

| | Quantity |
|---|---|
| Ingredient | (mg/unit) |
| Oxcarbazepine | 600.0 |
| (Median particle size 20.8 μm) | |
| Microcrystalline cellulose | 131.2 |
| Hydroxypropyl methyl cellulose | 16.8 |
| Poloxamer 188 | 20.0 |
| Cross-linked | 40.0 |
| polyvinylpyrrolidone | |
| Colloidal silicon dioxide | 3.2 |
| Magnesium stearate | 8.8 |
| Total | 800.0 |

1. Microcrystalline cellulose (about half the quantity) and hydroxy propyl methylcellulose were sifted through sieve; and mixed with oxcarbazepine for about 10 minutes to make a uniform blend.
2. Poloxamer 188 was dispersed in water and dry blend of step 1 was granulated.
3. The wet mass of step 2 was dried in fluidized bed dryer.
4. The dried material of step 3 was passed through sieve.
5. Cross linked polyvinyl pyrrolidone, colloidal silicon dioxide and microcrystalline cellulose (rest of the quantity) were sieved. These were then mixed with the dried material of step 4.
6. Magnesium stearate was passed through sieve and mixed with the material of step 5.
7. Lubricated blend of step 6 was compressed.

### Example 4

| | Quantity |
|---|---|
| Ingredient | (mg/unit) |
| Oxcarbazepine | 600 |
| (Median particle size 20.86 μm) | |
| Microcrystalline cellulose | 131.2 |
| Hydroxypropyl methyl cellulose | 16.8 |
| Sodium lauryl sulphate | 20.0 |
| Cross-linked polyvinylpyrrolidone | 40.0 |
| Colloidal silicon dioxide | 3.2 |
| Magnesium stearate | 8.8 |
| Total | 800 |

1. Microcrystalline cellulose (about half the quantity) and hydroxy propyl methylcellulose were sifted through sieve; and mixed with oxcarbazepine for about 10 minutes to make a uniform blend.
2. Sodium lauryl sulphate was dissolved in water and dry blend of step 1 was granulated.
3. The wet mass of step 2 was dried in fluidized bed dryer.
4. The dried material of step 3 was passed through sieve.
5. Cross linked polyvinyl pyrrolidone, colloidal silicon dioxide and microcrystalline cellulose (rest of the quantity) were sieved through #44 BSS. These were then mixed with the dried material of step 4.
6. Magnesium stearate was passed through sieve and mixed with the material of step 5.
7. Lubricated blend of step 6 was compressed.

### Example 5

| | Quantity |
|---|---|
| Ingredient | (mg/unit) |
| Oxcarbazepine | 600.0 |
| (Median particle size 20.86 μm) | |
| Microcrystalline cellulose | 131.2 |
| Hydroxypropyl methyl cellulose | 16.8 |
| Gelucire 44/14 | 60.0 |
| Cross-linked polyvinylpyrrolidone | 40.0 |
| Colloidal silicon dioxide | 3.2 |
| Magnesium stearate | 8.8 |
| Total | 800.0 |

1. Microcrystalline cellulose (about half the quantity) and hydroxy propyl methylcellulose were sifted through (44 BSS) sieve; and mixed with oxcarbazepine for about 10 minutes to make a uniform blend.
2. Gelucire was dispersed in water and dry blend of step 1 was granulated.
3. The wet mass of step 2 was dried in fluidized bed dryer.
4. The dried material of step 3 was passed through sieve.
5. Cross linked polyvinyl pyrrolidone, colloidal silicon dioxide and microcrystalline cellulose (rest of the quantity) were sieved through #44 BSS. These were then mixed with the dried material of step 4.
6. Magnesium stearate was passed through sieve and mixed with the material of step 5.
7. Lubricated blend of step 6 was compressed.

The oxcarbazepine tablets of Examples 1-5 were subjected to dissolution in 1% sodium lauryl sulphate in water according to the procedure described in the United States Pharmacopoeia XXIII, Apparatus USPII (Paddle)@ 50 rpm. A comparative dissolution profile with Trileptal®-600 mg (commercially available tablets of Novartis) is given in Table 1.

TABLE 1
Dissolution profiles of the oxcarbazepine tablets (prepared
by examples 1-5) in comparison with “Trileptal ®”,
in 1% aqueous sodium lauryl sulphate solution at 37° C./
50 rpm using apparatus USP II (Paddle)/900 ml.

| Oxcarbazepine | | | | |
|---|---|---|---|---|
| Tablets of | 10 min. | 30 min. | 45 min. | 60 min. |
| % drug release | 62.3 | 79.0 | 84.4 | 85.4 |
| for Example 1 | | | | |
| % drug release | 42.4 | 80.7 | 85.2 | 86.5 |
| for Example 2 | | | | |
| % drug release | 60.8 | 77.5 | 80.8 | 81.0 |
| for Example 3 | | | | |
| % drug release | 23.7 | 62.9 | 75.5 | 82.1 |
| for Example 4 | | | | |
| Example 5 | 39.4 | 73.3 | 78.5 | 81.4 |
| % drug release | 49.8 | 79.8 | 82.2 | 84.2 |
| for Novartis | | | | |
| (Trileptal ®) | | | | |

### Examples 6-10

The tablets of Examples 1-5 were further coated with a coating composition comprising:

| | |
|---|---|
| Hydroxy propyl cellulose | 12.0 mg |
| Talc | 12.0 mg |
| Iron oxide yellow | 0.50 mg |
| Water | q.s. |

Hydroxy propyl cellulose, talc and iron oxide yellow were dispersed in water and coated onto the tablets of Examples 1-5.

### Example 11

| | Quantity |
|---|---|
| Ingredient | (mg/5 ml) |
| Oxcarbazepine | 301.81 |
| (Median particle size 20.86μ) | |
| Microcrystalline cellulose and | 50.00 |
| sodium carboxymethyl cellulose | |
| Hydroxy ethyl cellulose | 150.00 |
| Aerosil 200 | 25.00 |
| Polyoxyl 8 stearate | 10.00 |
| Methyl paraben | 9.00 |
| Propyl paraben | 1.00 |
| Sorbic acid | 1.50 |
| Propylene glycol | 250.00 |
| Sodium saccharin | 10.00 |
| Ascorbic acid | 25.00 |
| Sorbitol solution | 1250.00 |
| Purified water | q.s. |
| Total | Up to 5 ml |

1. Avicel RC 591 was dispersed in water using colloid mill.
2. Polyoxyl 8 stearate was added in warm water and added to dispersion of step 1 under homogenization.
3. Sorbitol solution was added to dispersion of step 2.
4. Oxcarbazepine was dispersed in dispersion of step 3.
5. Hydroxy ethylcellulose was dispersed in water and transferred to dispersion of step 4.
6. Methyl paraben, propyl paraben and sorbic acid were dissolved in propylene glycol and transferred to suspension of step 5.
7. Ascorbic acid was dissolved in water and transferred to suspension of step 6.
8. Sodium saccharin was dissolved in water and transferred to suspension of step 7.
9. Aerosil was dispersed in suspension of step 8 and homogenized till a uniform suspension was formed.

While the present invention has been described in terms of its specific embodiments, certain modifications and equivalents will be apparent to those skilled in the art and are intended to be included within the scope of the present invention.

We claim:

1. Oxcarbazepine having a median particle size of from about 14 μm to about 30 μm.

2. A pharmaceutical dosage form for oral administration comprising oxcarbazepine having a median particle size of from about 14 μm to about 30 μm.

3. The dosage form of claim 2, wherein the oxcarbazepine has a median particle size of from about 14 μm to about 25 μm.

4. The dosage form of claim 2, wherein the dosage form comprises a solid or a liquid dosage form.

5. The dosage form of claim 4, wherein the solid dosage form comprises one or more of a tablet, a capsule, and a granulate.

6. The dosage form of claim 5, wherein the tablet dosage form is coated.

7. The dosage form of claim 5, further comprising one or more pharmaceutically acceptable excipients comprising one or more of surfactants, diluents, binders, disintegrants, lubricants, glidants, and coloring agents.

8. The dosage form of claim 4, wherein the liquid dosage form comprises one or both of a solution or a suspension.

9. The dosage form of claim 8, further comprising one or more pharmaceutically acceptable excipients comprising one or more of solvents, antioxidants, suspending agents, preservatives, surfactants, sweeteners, and flavoring agents.

10. A process for preparing a solid dosage from of oxcarbazepine, the process comprising:
mixing oxcarbazepine having a median particle size of from about 14 μm to about 30 μm with other pharmaceutical excipients to form a blend; and
forming the blend into a solid dosage form.

11. The process of claim 10, wherein forming the blend into a solid dosage form comprises forming a tablet, capsule or granulate.

12. The process of claim 10, wherein the mixing comprises dry granulation, wet granulation or direct compression.

13. The process of claim 12, wherein the wet granulation is carried out with a granulating fluid.

14. The process of claim 12, wherein the wet granulation is carried out with a solution or dispersion of a surfactant.

15. The process of claim 12, wherein the wet granulation is carried out with a solution or dispersion of a binder.

16. The process of claim 12, wherein the dry granulation is carried out by roller compactor or slugging.

17. A process for preparing a liquid dosage form of oxcarbazepine, the process comprising:
a) dispersing a suspending agent in water;
b) dispersing the oxcarbazepine having a median particle size of from about 14 μm to about 30 μm in the dispersion of step a); and
c) homogenizing.

18. The process of claim 17, wherein the suspending agent comprises one or more of polysaccharides, a mixture of cellulose and xanthan gum, a mixture of polyethylene glycol and sodium carboxymethyl cellulose, a mixture of xanthan gum and pregelatinized starch, a mixture of microcrystalline cellulose and sodium carboxymethyl cellulose, and dispersed silicon dioxide.

19. The process of claim 17, further comprising one or more pharmaceutically acceptable excipients comprising one or more of solvents, antioxidants, preservatives, surfactants, sweeteners, and flavoring agents.

20. A method of treating partial seizures in adults with epilepsy and as an adjunct therapy for treating partial seizures in children ages 4-16 with epilepsy, the method comprising orally administering to an adult or a child in need thereof a dosage form comprising oxcarbazepine having a median particle size of from about 14 μm to about 30 μm.
